(19) United States
(12) Patent Application Publication
(10) Pub. No.: US 2024/0344150 A1
(43) Pub. Date: Oct. 17, 2024

(54) METHODS FOR DETERMINING ANIMAL ANCESTERY AND FOR CONSERVATION THEREOF

(71) Applicant: Victory Genomics, Inc., Wilmington, DE (US)

(72) Inventor: Mustafa Khokha, New Haven, CT (US)

(21) Appl. No.: 18/635,256

(22) Filed: Apr. 15, 2024

Related U.S. Application Data
(60) Provisional application No. 63/496,029, filed on Apr. 14, 2023.

(51) Int. Cl. C12Q 1/6888

(52) U.S. Cl. CPC C12Q 1/6888; C12Q 2600/124

(57) ABSTRACT

Systems and methods for identifying the breed and sub-breed of in particular large animal, in particular a horse, and generating a breeding plan for producing foals with less risk and greater likelihood of successful birthing and achievement of healthy offspring. In one embodiment, the systems and methods include a genetic analysis that identifies for a particular horse the breed and sub-breed of that horse and ranks that horse has a function of inbreeding percentage. Further, the horse may be assigned for breeding and the methods described herein provide a mate from the breed or sub-breed that is selected to improve the heterozygosity of the foal.

### CLAIM OF PRIORITY

This case claims priority to earlier filed U.S. Provisional Patent Application 63/496,029, entitled METHODS FOR DETERMINING ANIMAL ANCESTRY AND FOR CONSERVATION THEREOF, filed Apr. 14, 2023 and naming Mustafa Khokha as inventor, the contents of which are incorporated herein by reference in their entirety.

### BACKGROUND

Today horse breeding is done through conventional animal husbandry techniques with minimal support arising from genetic science. Although scientists have developed some genetic testing for improved animal breeding, such as those for canines disclosed in US Patent Application No. US2006/0008815 entitled Compositions, Methods, And Systems For Inferring Canine Breeds For Genetic Traits And Verifying Parentage Of Canine Animals, which discusses gene association analyses and the use of single nucleotide polymorphisms to determine traits, parentage identity and breed of dogs, largely animal breeding remains dominated by traditional husbandry practices.

Breeds are largely determined by the appearance and parentage, if known, of the animal. Breeds are also associated with certain behavior and risks of disease. For example, large animals, like horses, are in large part identified by their breed, such as Thoroughbred, Arabian, Palomino, Clydesdale and other breeds. Certain breeds have certain characteristics, including morphological and genetic characteristics. Thoroughbreds are powerful animals capable of running at remarkably high speeds for relatively short distances, typically two miles or less. Clydesdales present size and musculature and are very capable of pulling the types of loads such as large wagons, that other horses would be incapable of moving. The Arabian breed is powerfully built, has high tail carriage and is capable of traversing notably long distances such as 100 miles during the course of a day. The characteristics of these different breeds turns in part on selection of certain qualities that help the breeds achieve particular levels of performance. In any case, traditional techniques for determining breed and sub-breed largely include checking parentage if known and comparing a particular horse to a breed or sub-breed standard set by a particular organization. That standard will commonly include physical traits, such as size, head shape, and color, that the animal is to meet to be deemed a member of a particular breed or sub-breed.

The success of a horse breed, such as the Arabian, can lead to several sub-breeds. For the Arabian horse, sub-breeds may include the Crabbet, Egyptian and the Turkish sub-breeds. Each sub-breed of Arabian horse has different morphological characteristics and dispositions. The Crabbet sub-breed is heavier and more robust than the Egyptian sub-breed. The Turkish sub-breed can vary from these as well. All this can be reflected in the standard for the sub-breed and the standard can be recorded and published for use by subsequent breeders and buyers.

Each sub-breed of horse is important to owners and members of industry that rely on the success of their sub-breed. This can include sportsman that raise and race a particular sub-breed, and others that use Arabians for more traditional purposes such as transportation. As such, there is a need to be able to determine the ancestry of an animal and its membership in a sub-breed with more accuracy than provided by assessing the physical traits of the animal against published standards for body-type for the sub-breed.

As successful as horse breeding has been, there also remains a concern that the vitality of sub-breeds may be fragile. Breeding large animals, whether horses, camels, cattle, elephants, or otherwise is an expensive and risky proposition. Inbreeding, infertility, and the promulgation of disease are always of concern. The success of a sub-breed turns on the ability to reliably produce healthy off-spring.

Accordingly, there is a need in the art for systems and methods that can more accurately and safely improve identification of a large animal's breed and sub-breed and for conservation of animal breeds including animal sub-breeds.

### SUMMARY OF THE INVENTION

The systems and methods described herein include, inter alia, systems and methods for identifying the breed and sub-breed of large animals, in particular a horse, but other animals may be treated with the systems and methods described herein including camels and cattle. In any case the systems and methods generate a breeding plan for producing foals, calves and juvenile members of other species, with less risk and greater likelihood of successful birthing and achievement of healthy offspring. Although the systems and methods described herein are addressed primarily to large animals, it will be understood that these may also apply to smaller animals, including birds, canines and others.

In one embodiment, the methods described herein can determine the ancestry of a horse, including determining whether the horse is by genetics, a member of a particular sub-breed, such as a Crabbet Arabian, to provide higher accuracy breed or sub-breed identification as compared to an assessment of body-type standards. In one further aspect, the systems and methods described herein include a genetic analysis that identifies for a particular large animal, such as a horse, the breed and sub-breed of that horse. A sub-breed may be understood as a subset of a breed where that subset has a known standard. For example, the breed of Arabian horses has sub-breeds such as the Egyptian sub-breed and these sub-breeds are associated with respective standards that set forth the morphology and optionally certain genetic characteristics of the sub-breed, such as for the Egyptian sub-breed of Arabians. Breeds and sub-breeds of cattle are extensive, and for example include Japanese Black, Japanese Brown, and Mishima and standards exists for such cattle sub-breeds. In general, sub-breeds are understood as generally fertile lines of the breed and as such are capable of propagating the breed such that breeding within the sub-breed produces a juvenile of the same sub-breed and having the same morphological characteristics, often for different purposes such as endurance or speed, set forth in the standard as other members of the sub-breed. The systems and methods described herein provide a more genetic based and quantitative assessment of the characteristics of a sub-breed that conventional morphology standards. Such a quantitative assessment can provide a step toward improved sub-breed breeding practices for conservation of the sub-breed.

To this end and in one aspect, disclosed herein are methods that, in certain embodiments, provide methods for conserving a sub-breed of a large animal. These methods may collect genetic sequence data from a population of the large animal that are known to be in the sub-breed. The population typically includes cohorts of the large animal, each large animal in a cohort known to be in one of the multiple breeds and sub-breeds of large animals so that the population includes large animals of different breeds and sub-breeds. This provides genetic sequence data that allows for analyzing the different breeds and sub-breeds relative to each other.

The method then performs principal component analysis to develop a measure of genetic variation between a large animal member of the population of large animals and a reference genome, wherein the dimension of the principal component analysis is selected to allow for clustering analysis. The reference genome typically is the genetic data associated with a representative member of the large animal. Comparison of respective animals against this reference genome gives a data about the genetic differences between members of the breed, including members of sub-breeds versus the reference genome.

The method performs a cluster analysis to determine whether the principal component analysis yields clusters that are capable of distinguishing between breeds and sub-breeds with a threshold level of accuracy, and selects a respective one of the large animals in a cohort of a certain sub-breed for breeding and determining a level of heterogeneity. The method selects a mate, from the same sub-breed, for the selected large animal based on a heterogeneity profile of the selected large animal to find a mate having a genomic profile selected to produce healthy offspring with a selected heterogeneity.

In optional embodiments, the method collects genetic sequence data from a population of the large animals that are known to be in the sub-breed and this includes identifying a population of the large animals by comparing morphological characteristics of a population of large animal against morphological standards set of a sub-breed of the large animal.

In further embodiments, collecting genetic sequence data from a population of the large animals that are known to be in the sub-breed includes identifying a population of the large animals by comparing parentage breed data of a population of large animal against wherein the parentage breed data includes information about the morphological characteristics of ancestors of the large animal as compared against morphological standards set of a sub-breed of the large animal.

In further embodiments, the method collects genetic sequence data from a population of the large animals that are known to be in the sub-breed by identifying a population of the large animals through comparing genomic sequence data of a population of large animals against genomic sequence data associated as a standard set for a sub-breed of the large animal.

Typically, the method analyzes the data by performing principal component analysis on a measure of genetic variation between a member large animal of the population of large animals and a reference genome by comparing gene sequence data for the member large animal population against gene sequence data of the reference animal and identifying differences between the two sequences. The method may perform cluster analysis by generating a dendrogram to determine a relationship between members of sub-breeds for allocating animals to clusters. Optionally, the method may perform a cluster analysis includes a nearest neighbor analysis, or some other suitable analysis.

Typically, selecting a respective one of the large animals in a cohort is done by analyzing the genome of the respective large animal, considering the paternal and maternal genetic data and making a fractional analysis of identity of paternal and maternal genetic data to measure a level of inbreeding.

Selecting a mate to find a mate having a genomic profile selected to produce healthy offspring with a selected heterogeneity may include selecting a mate having greater heterogeneity at a select sequence location than the selected large animal.

Selecting a mate, from the same sub-breed, for the selected large animal based on a heterogeneity profile of the selected large animal to find a mate may include selecting a large animal having a genomic profile selected to produce healthy offspring capable of breeding.

In some embodiments, the method generates a B-allele plot for the selected large animal to provide a heterogeneity profile for the selected large animal and generating a B-allele plot for the candidate mate to provide a heterogeneity profile for the candidate mate and determining the quality of the candidate mate for as a function of the similarity of the heterogeneity profiles of the candidate mate and the selected large animal.

The methods described herein may be employed on many large animals including horses, camels and cattle.

The methods may generate a graph to provide a visual exploration of a horse's genetic relationship to other members of a sub-breed and breed. Further, the methods may develop a B plot of all 32 chromosomes of a particular horse under review, showing areas of homozygosity and heterozygosity. The method may determine for a horse in a population of horses of a breed or sub-breed, a genetic inbreeding coefficient representative of an inbreeding percentage of a horse and a population of horses. Further, a horse may be designated for breeding and the methods described herein provide a mate from the breed or sub-breed that is selected to improve the heterozygosity of the foal. Such selective breeding provides conservation of the breed or the sub-breed when that breed or sub-breed is fragile and tends toward loss of heterozygosity and promulgation of genetic disease.

### BRIEF DESCRIPTION OF DRAWINGS

The foregoing and other objects and advantages of the systems and methods described herein will be appreciated more fully from the following further description thereof, with reference to the accompanying drawings wherein, the Figures set forth in this application are for illustration and disclosure purposes and are not to be understood as limiting in any way.

FIG. 1 depicts the body-type of a sub-breed of Arabian horse deemed the Crabbet Arabian;

FIG. 2 depicts the body-type of a sub-breed of Arabian horse deemed the Egyptian Arabian;

FIG. 3 depicts an example of a body-type standard set by a sub-breed standard organization, in this case for the Egyptian Arabian sub-breed;

FIG. 4 depicts a principal component analysis (PCA) graph of the DNA and gene sequence data of multiple animals and a clustering of data to identify that the PCA analysis of genetic data can delineate breed and sub-breed for horses;

FIG. 5 depicts a series of violin graphs indicating a genetic inbreeding coefficient and density of those coefficients for animals within a breed or sub-breed;

FIG. 6 and FIG. 7 depict side-by-side analyses of a breed of horse and a sub-breed of that horse;

FIG. 8 depicts a graph of a horse showing the B Allele plot for 32 chromosomes of the horse in this example;

FIG. 9 depicts one process for determining breed and sub-breed of an animal by developing a database of DNA and genetic information performing an analysis of that data to identify genetic data indicative of breed and sub-breed;

FIG. 10 depicts one process for identifying a measure of inbreeding for a breed or sub-breed of animal; and

FIG. 11 depicts a process to identify a breeding partner for that animal to provide a conservation breeding partner suitable for breeding in a manner to conserve the breed or sub-breed.

### DETAILED DESCRIPTION

To provide an overall understanding of the invention, certain illustrative embodiments will now be described. However, it will be understood by one of ordinary skill in the art that the systems and methods described herein can be adapted and modified for other suitable applications and that such other additions and modifications will not depart from the scope hereof.

Although the systems and methods described herein may be employed with any large animal and optional smaller animals as well, the example illustrations will be largely drawn to analysis of sub-breeds of horses where there is a need to conserve the sub-breed and the quantitative measures employed herein allow for improved breeding to keep the sub-breed healthy and fertile without having to add cross-breeding with other sub-breeds or breeds.

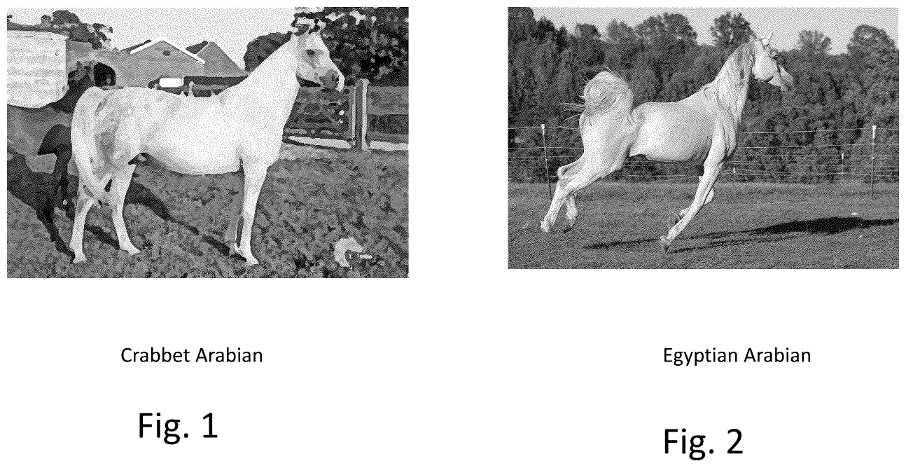

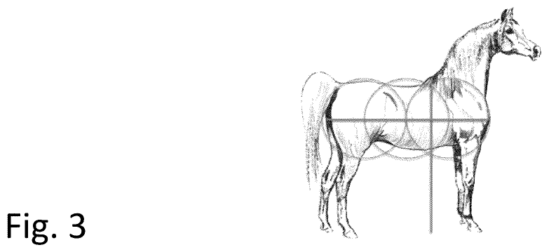

FIGS. 1 and 2 depict examples of the body-type of sub-breeds of Arabian breed horses. As one can tell from reviewing FIGS. 1 and 2 there are differences between the sub-breeds which are determinable by viewing the animals and considering physical appearance of the animals. FIG. 1 depicts a Crabbet Arabian sub-breed and FIG. 2 depicts the Egyptian Arabian sub-breed. The Crabbet breed of FIG. 1 is more robust and thicker around its core. The Egyptian Arabian is more delicately built and has a more pronounced scallop face. The sub-breeds of the horse are often set by organizations that identify a body-type standard. FIG. 3 depicts in an overview manner and graphically an example body-type standard for the Egyptian Arabian sub-breed. This standard can identify proper proportions for shoulders, head size, how the tails should be carried, and other similar morphological information. Parentage and Body-type standards are the prevailing method of determining breed and sub-breed for horses.

The genome of horses has been studied. In particular, a horse genome for the horse Twilight, a Thoroughbred mare, has been identified and is provided in a public database. Information about the genome for Twilight may be found at T Raudsepp et al.; Ten Years of the Horse Reference Genome: Insights Into Equine Biology, Domestication and Population Dynamics in the Post-genome Era; Anim Genet.; 50(6):569-597 (2019 December). The genome information provides most of the sequence for the particular horse Twilight with some gaps at known gap locations. From time to time, the genome data is improved and new versions of the reference genome are made available. For the examples set out in this application version 3 of the Twilight reference genome was employed.

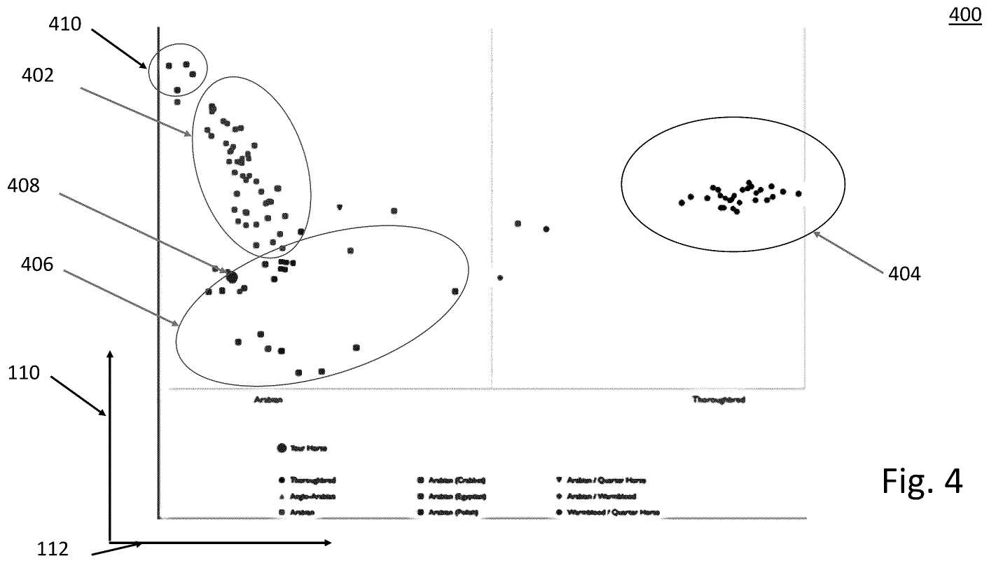

As populations of sub-breeds may be fragile and at risk, the methods illustrated herein in the following figures describe a method for conserving a sub-breed of a large animal, which in this example is sub-breeds of horses. A will be discussed in this example as to horse sub-breeds, the method will collecting genetic sequence data, often whole genome, although the Y chromosome may be left out of the PCA operation. The genomic data is collected from a population of the large animals that are known to be in the sub-breed. So in this example, the members of the sub-breeds set out in FIGS. 1-3 will be known by morphology and parentage. These sub-breeds make us a cohort population of the large animal. Genetic data from this cohort can be tested against a reference genome. As will be shown by FIG. 4 for example, PCA analysis will elucidate the range of genetic similarity for this cohort and will set the parameters for a clustering analysis that may be employed to place new members into the genetically defined clusters found by these methods. In this way, PCA yields clusters that are capable of distinguishing between breeds and sub-breeds with a threshold level of accuracy. That threshold can be set based on the rigor with which one chooses to apply and one's willingness to accept overlap of cohorts and isolation of members. In one technique, one sets the level of accuracy to require that at least 60%, 75%, 95% or some other accuracy level that members of a sub-breed cohort have been placed into that sub-breed cluster by using morphology and parentage (that is by use of typical qualitative assessment), fall within the cluster for that cohort, and optionally that is there is no overlap, or overlap below a set threshold, with any or at least certain other cohort clusters. Parentage data can be determined by analysis of breeding records, and optionally by DNA testing of the foal against genomic data of the purported parents. In any case, members of a cohort should sort into that cluster and the level of accuracy of that sort can be set by the of the many known techniques for determining the level of accuracy can be used and any suitable technique can be used.

FIG. 4 depicts a dimensionless graph produced using principal component analysis (PCA) with dimensionless axes 110 and 112. FIG. 4 depicts breed heritage and relationships and provides a visual representation of a horse's genomic relationship to other breeds. In one aspect, FIG. 4 provides a map that classifies horses into different breeds and types based on their DNA. By comparing the genetic data of an individual horse to known breed profiles, it shows a horse's genetic relationship to other horses in a sub-breed, such as Crabbet's (and part-Crabbet), to other Arabian types, and the genomic distance from thoroughbreds.

The PCA works with the genetic sequence data of the sampled animal population. The genetic sequence data may be understood to refer to the detailed information about the DNA sequences of these large animals. Optionally, the systems and methods described herein determine the whole genome or essentially the whole genome. In some embodiments, the systems and method described herein do not employ the Y chromosome, the sex chromosome, to allow for more useful comparison of genetic data in a breed across genders. The genetic sequence data is employed herein for, among other things, identifying specific genes and their variants, known as alleles, which can influence traits of interest in animal breeding programs. Typically, in breeding, genetic sequence data is used in two main ways. First, the data is employed from a statistical perspective where genomic data are treated as large sets of markers of ancestry. Breeders use these markers to estimate breeding values without necessarily understanding the exact function of each marker. This approach, known as genomic selection, has become a mainstream part of animal breeding. Second, the data is employed from a sequence perspective such that the genomic data may be employed as a long string of amino acids A, C, G and T are this string is a source of causative variants, and the goal is to identify specific sequences in the DNA that directly cause variations in traits, and lead to indications of a sub-breed. Once identified, these causative variants can be used to make more informed breeding decisions. Techniques for determining the genomic data, including whole genome, of large breed animals have been discussed in, for example, Rivas V N, Magdesian K G, Fagan S, Slovis N M, Luethy D, Javsicas L H, Caserto B G, Miller A D, Dahlgren A R, Peterson J, Hales E N, Peng S, Watson K D, Khokha M K, Finno C J. A nonsense variant in Rap Guanine Nucleotide Exchange Factor 5 (RAPGEF5) is associated with equine familial isolated hypoparathyroidism in Thoroughbred foals. PLoS Genet. 2020 Sep. 28; 16(9), the content of which is incorporated by reference herein. Such techniques may be employed by the systems and methods described herein to collect genome data from the animals in the population.

The map of FIG. 4 is generated using a principal component analysis (or PCA) done on the genome data of the various large animals, in this case horses. A principal component analysis was undertaken to reduce the dimensions of the data set, in this case thousands of base pairs, to two dimensions thereby enabling the generation of the graph (or map) 400 of FIG. 4. To create the graph, each horse genome from a population of horses of known breed or sub-breed was measured against the same reference genome. For this example, the reference genome was for Twilight, the Thoroughbred mare noted above. In practice, the genome of horse, such as an Arabian breed horse, would be compared to the reference genome for Twilight. Deviations from the reference genome for Twilight are measured and principal component analysis is used to reduce those measurements to two orthogonal criteria. Principal component analysis is well known in the art.

The systems and methods described herein may employ any suitable techniques for Principal Component Analysis (PCA). PCA, as is generally known to those of skill in the art, is a data analysis technique that allows for visualizing and simplifying complex genomic data. Scientists often use PCA to explore relationships between samples, identify patterns, and reduce dimensionality. Typically, one performs Data Preparation where the scientists start with a dataset containing genomic information (e.g., gene expression levels, SNP data, or other features). The gene expression data for the representatives of the sub-breed may be obtained by collecting for example blood samples from those large animals identified as within the sub-breed. For example, one may identify a plurality of horses that have been identified as Crabbet Arabian horses by people skilled in the area of Arabian horses. Such identification is often accomplished by comparing the physical traits of the horse against the standards set of the Crabbet sub-breed, such as tail carriage and torso girth. Additionally, and optionally, the parentage of a horse may be considered and this may involve checking that both parents of the foal in question, likely now a grown horse, where identified as members of the sub-breed, such as the Crabbet sub-breed. Typically, a data matrix usually has samples (individuals) as rows and features (genes, variants, etc.) as columns. In a next act, one will compute genetic distances, often by using tools such as PLINK, scientists calculate genetic distances (the extent of genetic differences) between samples based on the genomic data. These distances may represent how similar or dissimilar samples are in terms of their genetic makeup. The process may then undertake Multidimensional Scaling (MDS) of the genetic distance matrix to create a lower-dimensional representation. This transforms the high-dimensional data into a smaller set of coordinates (principal components) while preserving pairwise distances. The result is typically a set of eigenvalues and eigenvectors. The eigenvalues represent the proportion of variance explained by each principal component. The eigenvectors (also often called loadings) indicate the contribution of each original feature to the principal components. With this, the method may undertake a PCA plot creation where the first principal components (PCs) capture most of the variation in the data. Scientists plot samples in the space defined by the top PCs, which in certain examples herein are two dimensions. The x-axis may corresponds to PC1 (highest variance), and the y-axis to PC2 (second highest variance). Each sample is represented as a point in this reduced-dimensional space. Samples that are genetically similar cluster together, while dissimilar samples are farther apart. The PCA plots may identify patterns, clusters, or outliers and help identify the genetic variation seen in a sub-breed, or the different sub-breeds of the breed. Thus, as will be discussed with reference to FIG. 4, one can see the range of genetic variation from the reference animal that Crabbet Arabians have versus Egyptian, as well as the differences between breeds such as Thoroughbreds versus the sub-breeds of Arabian. As FIG. 4 shows, given the data set of gene sequences, one may also find the PCA gene differences for mixed breed animals, such as the Arabian/Quarter horse mix and the Anglo-Arabian mix breed.

The PCA plots may help reveal underlying population structure, genetic ancestry, or subgroups such as sub-breeds within the data. The plots may also aid in identifying potential confounding factors or batch effects. In any case, the FIG. 4 and such plots provides a quantitative basis for identifying sub-breeds within a population of large animals, in this case horses.

As can be seen, each dot in the map of FIG. 4 indicates the genomic difference from Twilight of a particular horse, where that horse is a member of the population of horses tested and compared to the appropriate reference horse, in this case Twilight. From the map 400, one can see that a population of Thoroughbred horses (noted on the x-axis) appear as cluster 404. This identification of the cluster 404 as encompassing Thoroughbreds was found by determining the differences between a subset of horses and the reference genome for Twilight. Those genomic differences were plotted using principal component analysis onto the two-dimensional graph depicted in FIG. 4. Cluster analysis, using any of the known suitable techniques, of the plotted genomic difference data was performed In some embodiments, the method may perform cluster analysis by generating a dendrogram to determine a relationship between members of sub-breeds for allocating animals to clusters, or optionally the method may perform employ a nearest neighbor analysis, or some other suitable analysis. As can be seen a group of horses clustered within the area defined by cluster 404. Analysis of the data within that cluster 404 revealed that the genomes in this cluster 404 are all associated with large animals that have been identified as of the Thoroughbred breed. Thus, the indication of the breed Thoroughbred in FIG. 4 on the x-axis and below the cluster 404 is data that was derived from this technique of using principal component analysis and clustering. That is the cluster of 404 was found and it was then determined that the horses within that cluster 404 were Thoroughbreds. This established the efficacy of genomic analysis of horse breeds as determined by determining genomic differences from a reference horse genome.

FIG. 4 further depicts data collected from horses associated with the Arabian breed. These data points are depicted as squares within graph 400. Within these data points for the Arabian breed, are data points for the Arabian sub-breeds Crabbet, Egyptian and Polish.

Cluster 402 encompasses horses identified as Arabian, but not part of a sub-breed. Cluster 406 encompasses horses identified as from the Crabbet sub-breed, as well as encompassing some others, including some Polish Arabians. Cluster 406 shows how the Crabbet DNA broadly clusters together, while remaining significantly separated from Thoroughbreds, and enjoying a closer genomic relationship to Polish lines. All the Arabian types are closely related, yet the modern Egyptian Arabian are set out in cluster 410 and are somewhat differentiated from the Crabbet.

FIG. 4 further depicts a data point 408. Data point 408 indicates the principal component analysis of genome data for a particular candidate horse understood as a Crabbet Arabian. As can be seen, the horse associated with data point 408 is in cluster 406 and is somewhat spaced away from the cluster 402. In certain embodiments, the separation of clusters where those clusters have been identified as associated with respective groups of breeds and sub-breeds, may indicate the effectiveness of the principal component analysis. In such cases, the effectiveness of the principal component analysis to reduce the genomic data of a horse to a two-dimensional representation maybe correlated with the ability of the PCA approach to separate into distinct clusters different breeds and sub-breeds of horses.

Further, FIG. 4 also depicts that certain horses may have pedigrees, that is a genomic makeup, that includes genetic material from two or more breeds or sub-breeds of horses. Thus, for example an Anglo Arabian, which is a sub-breed of Arabian that includes some Anglo, may have a genetic sequence that indicates the horse has genetic material from and Arabian breed and an Anglo breed. Such information may be employed to identify horses offered as members of a subgroup, but that have been bred to have genetic material from another breed or sub-breed. Through this approach a check of ancestry and membership in a sub-breed may be confirmed. Consequently FIG. 4 depicts that DNA and gene sequence data is a reliable way to determine the breed and sub-breed of a horse. As such breed and sub-breed do not necessarily need to be determined by visual comparison of the candidate animal to the standards identified by societies for that animal.

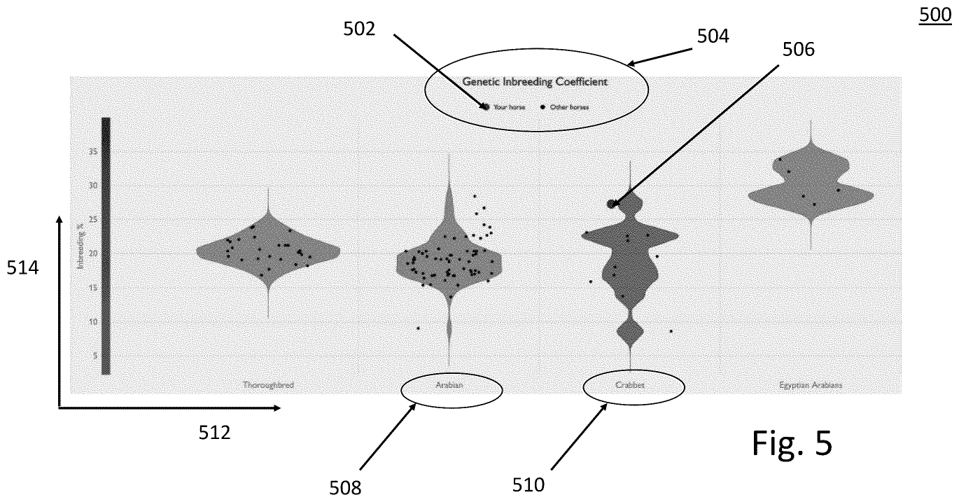

FIG. 5 depicts a graph 500 that includes four violin graphs indicating the percentage of inbreeding for different breeds and sub-breeds of horses analyzed. The Y-axis indicates the percentage of inbreeding. The X-axis indicates where groups of a certain breed or sub-breed of horses have similar levels of inbreeding. As such, the width of a respective graph within FIG. 5 graph 500 indicates and suggests the level of inbreeding that is most common for that breed or sub-breed.

The graph 500 of FIG. 5 can be understood as a breed conservation plot that shows visually the overall health of the Crabbet Arabian breed. It does so by an analysis of 5.5 million genetic variants across each individual horse genome, then compares this to many other horses of the same and different types and breeds. In doing this, the genetic inbreeding coefficient is determined, which is far more accurate than traditional pedigree-based inbreeding coefficients.

The breed conservation plot 500 of FIG. 5 shows the range of inbreeding across Crabbet Arabians, shown by the length of plot: from around 8% to about 30%; the width of the plot depicts where groups of Crabbets with similar levels of inbreeding cluster together; the overall position of the plots shows how Crabbets are faring compared to other breeds and types—Egyptians, for example, have a higher rate of inbreeding, which affect the conservation of the breed; and a particular horse's place within the breed, and it may be the horse being considered for breeding or purchase.

Analysis of FIG. 5 makes it readily apparent that the Egyptian Arabian sub-breed has a high percentage, relative to the breed and other sub-breeds, of inbreeding. In contrast the thoroughbred breed appears to have a lower percentage of inbreeding and the relative degree of inbreeding that is the amount of inbreeding that is the smallest for the analyzed group versus the largest for the analyzed group, is relatively compact and centered around a 20% number. In contrast the Arabian has a good average level of inbreeding but the spread is relatively large with a low number around 9% and a high of close to 30% in breeding.

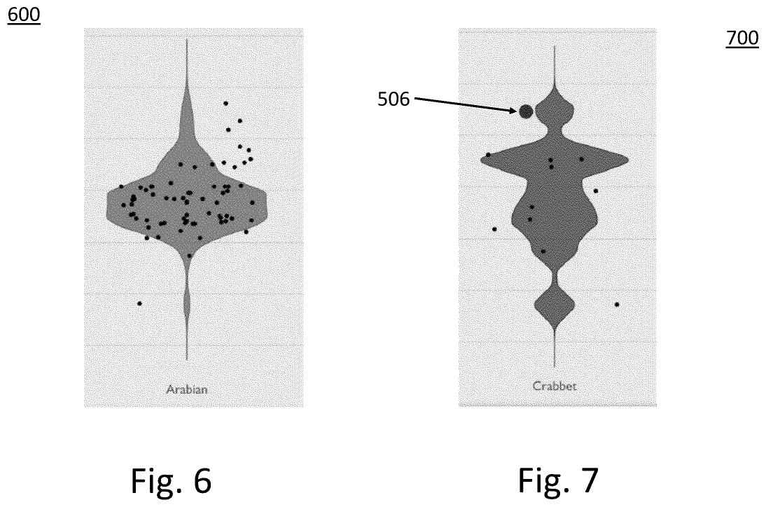

FIG. 6 and FIG. 7 show side-by-side comparisons of the plotted data for Arabians and for the sub-breed of Crabbet Arabian. FIG. 6 and FIG. 7 portray graphs shown in FIG. 5 but show those graphs in more detail. The side-by-side depiction of the Arabian and the Crabbet helps illustrate that the Crabbet Arabian sub-breed has a more fragile genetic makeup in that a greater portion of the horses in the sub-breed have higher levels of inbreeding percentages. This can make conservation of the breed, such as the Crabbet Arabian sub-breed complex and difficult as the pool of possible mates includes a substantial portion that have high levels of inbreeding percentages. High levels of inbreeding percentages will increase the likelihood that diseases will pass to foals.

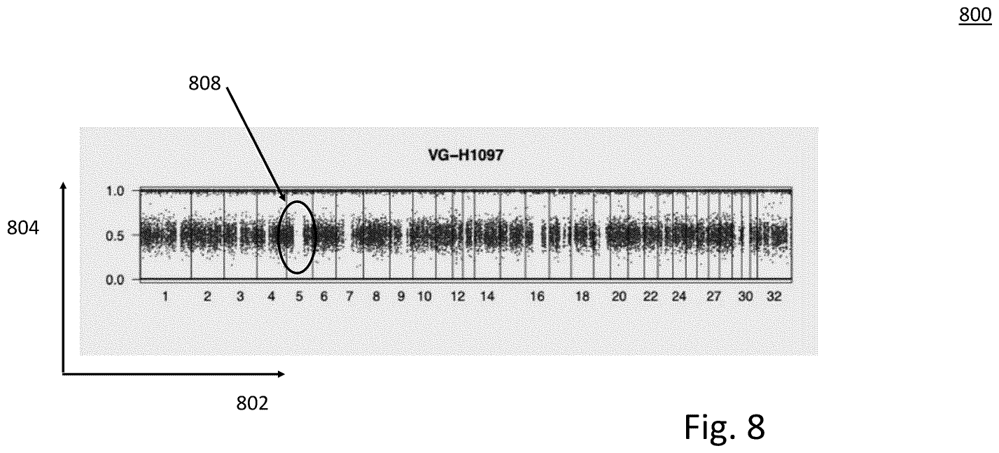

FIG. 8 depicts one example of a measure of inbreeding percentage using a B-Allele plot and a percentage evaluation of that plot that considers the alleles compared and the number of alleles that showed identity between both parents.

In particular, FIG. 8 shows a B-Allele plot for the 32 chromosomes of a horse. In this case the horse is identified by the label H1097. Each of the chromosomes is identified by number, on the X-axis of the graph, 1 through 32 with 32 as the gender gene. The Y-axis provides values from 0.0 to 1.0 with 0.5 marked halfway up the Y-axis. The Y-axis shows the result of a comparison for bases or segment of bases (alleles) at a genomic location (which is correlated by the x-axis to a chromosome) and whether the bases or segments are the same, indicating that the horse inherited the same base or segment from both parents. If analysis finds that the same allele came from both parents, a 1.0 is marked for that genomic location. If they are half different, the analysis marks a 0.5 at that location. Other levels of differences between the alleles may also be indicated and the range for the center band in FIG. 8 is about 0.4 to 0.6.

A horse's genome is made up of over 20,000 genes spread across 32 chromosomes. These genes have alleles that can be the same (homozygous) or different (heterozygous). An allele is generally understood as one of two or more versions of DNA sequence (a single base or a segment of bases) at a given genomic location. An individual inherits two alleles, one from each parent, for any given genomic location where such variation exists. If the two alleles are the same, the individual is homozygous for that allele. If the alleles are different, the individual is heterozygous. When there are too many alleles that are the same it can manifest as a disease, infertility, or a trait that can be harmful (e.g. behavior). The plot 800 presented in FIG. 8 shows areas in each of the analyzed horse's 32 chromosomes where the alleles are the same or different. This shows genetic inbreeding at a chromosome level.

FIG. 8 shows for each chromosome those gene sequences that are inherited from both the mare and the stallion. Those are indicated as dots along the line labeled 1.0. For example, turning to chromosome 5 it can be seen that the band around 0.5 for chromosome 5 has a large gap. In that large gap the gene sequences are common to both the mare and stallion. As such, FIG. 8 is a representation of the homogeneity and heterozygosity of the animal being tested. Homogeneity can be a good measure of the likelihood of having inherited diseases and is representative of inbreeding. For example, in humans there is typically 2 to 3% of homogeneity in a person. In a person who is the progeny of a consanguineous relationship, homogeneity can be 15 to 20%. It is understood that high levels of homogeneity can lead to infertility, diseases, malformations, and other disadvantages. In FIG. 8, for chromosome 5 the large center gap indicates thousands of genes that are common to the mare and the stallion. Genes having mutations coding for diseases and abnormalities that are undesirable will likely or more likely be expressed when that genetic material is passed to the foal from both the mother and the father.

In one aspect of the invention, conservation of a breed or sub-breed is improved by testing the homogeneity of a first animal in a group or subgroup and finding a mating pair whose homogeneity avoids the homogeneity pattern of the first animal. In this way, the likelihood of promulgating genetic diseases and other maladies during breeding is reduced.

So, for example returning to FIG. 7, it can be seen that the Crabbet Arabian sub-breed has a limited number of members and perhaps slightly elevated levels of homogeneity and therefore inbreeding. In a breeding process, a first animal in the Crabbet Arabian sub-breed may be selected. Homogeneity data for that Crabbet Arabian animal may be obtained. The sequences for that animal that have been inherited fully from the mare and stallion that produced that animal may be measured and considered. That consideration would include identifying those sections of the gene sequence, that is the gaps in the homogeneity analysis, and identifying a mating pair within the sub-breed of Crabbet Arabians that has homogeneity which is compatible in that it does not promulgate the gene sequences inherited from both the mayor and stallion of the first animal. In this way the breed or sub-breed may be conserved in that the genes that are producing the morphological and genetic makeup of the breed or sub-breed are maintained without incurring the high risk of promulgating genetic diseases to a foal. The foal therefore has a genetic vigor that is desirable and conforms to the genetic makeup of the Crabbet Arabian sub-breed.

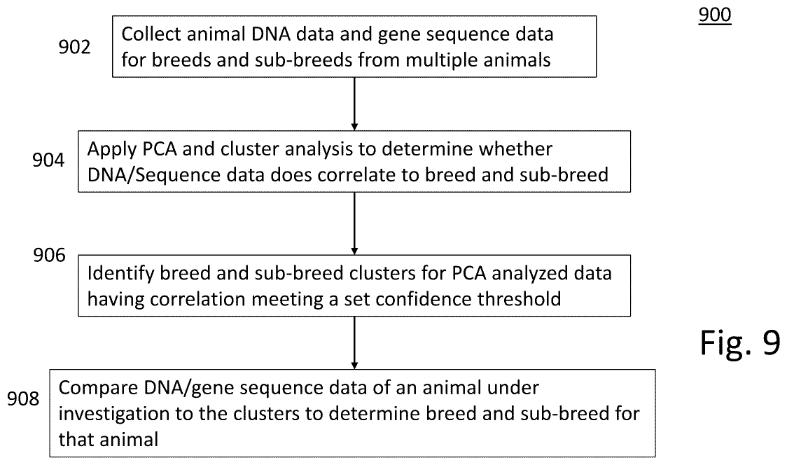

In another aspect, disclosed herein are methods for determining the breed or sub-breed of a particular animal. FIG. 9 depicts one such method. The process depicted 900 begins at a 902 where animal DNA is collected and DNA and gene sequence information for breeds and sub-breeds is collected and determined for multiple animals within a breed or sub-breed. The method then proceeds to step 904 wherein principal component analysis and cluster analysis is applied to the genetic data to determine whether the DNA and or sequence data does correlate to a breed or sub-breed. This can be done by performing PCA and cluster analysis and then determining whether or not the clusters are good representations of animals in a particular sub-breed or breed. The process 900 then may proceed to step 906 wherein the process identifies breed and sub-breed clusters for PCA analyzed data having correlation meeting a set confidence threshold. Any suitable confidence threshold may be applied, including ones that check that a sufficiently high, perhaps greater than 60%, of a particular sub-breed are encompassed by an cluster. That is the clusters maybe determined and tested against actual data wherein known animals having known breeds or sub-breeds are checked against the PCA and clustered data to make sure that the PCA and cluster process is effective to a set confidence threshold. Alternately, the confidence threshold can be set using P values or other types of statistically significant methods for determining the accuracy and effectiveness of an analytical technique.

The process 900 then proceeds to 908 wherein the process will compare DNA and gene sequence data of an animal under investigation to the clusters to determine the breed and sub-breed for that animal. Thus, when the breed or sub-breed is not known, or not confirmed for a particular animal the gene sequence information may be used with the information on a graph such as the graph depicted in FIG. 4 to determine whether the animal under investigation has a genetic makeup that falls within one of the clusters associated with the particular breed or sub-breed.

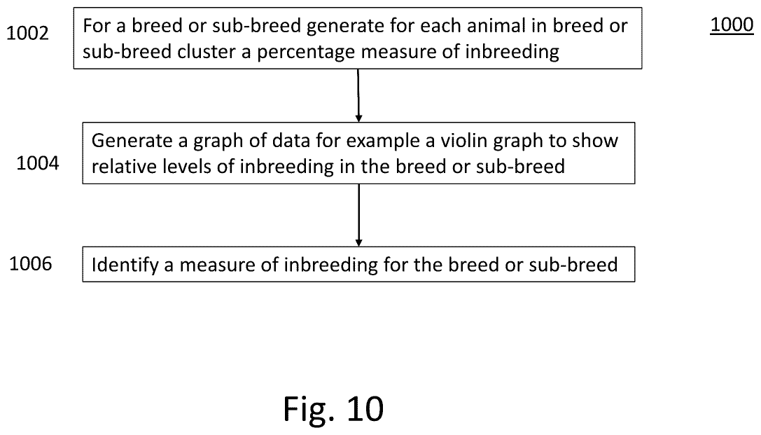

FIG. 10 depicts the process 1000 wherein for a breed or sub-breed a measure of inbreeding is determined. The process 1000 begins in 1002 by having the process for a breed or sub-breed generate for each animal in the breed or sub-breed cluster a percentage measure of inbreeding. As discussed above with respect to FIG. 8, one measure of inbreeding is to create a B allele plot that can be used to measure the homogeneity of the particular animal. The process then proceeds to 1004 wherein the process generates a graph of data, for example a violin graph, to show relative levels of inbreeding in the breed or sub-breed. The process then proceeds to 1006 wherein the process will identify a measure of inbreeding for the breed or the sub-breed. This measure which may be arranged give some indication of the relative fragility of the particular breed or sub-breed wherein fragility is understood to mean a question or concern about the likelihood that the population that exists can produce healthy offspring in this case foals.

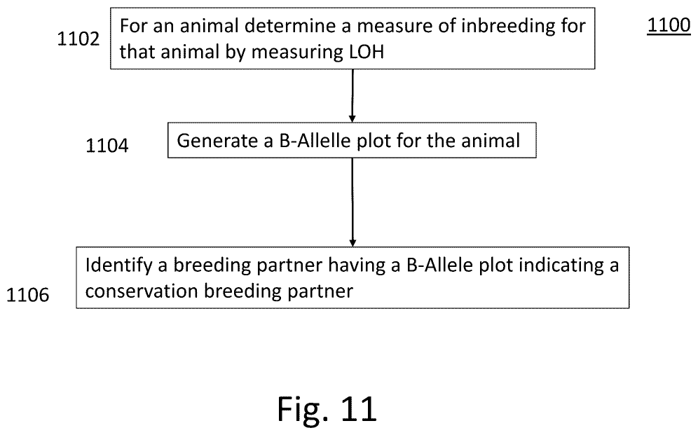

As the ability for a population of a breed or sub-breed to produce healthy offspring, the systems and methods described herein may be used to help conserve a breed or sub-breed. One method for this conservation is depicted in FIG. 11. The example depicted process may help select a respective one of the large animals in a cohort of a certain sub-breed for breeding and determining a level of heterogeneity, and select a mate, from the same sub-breed, for the selected large animal based on a heterogeneity profile of the selected large animal. The selection process is intended to provide health, fertility and avoid cross-breeding for the sub-breed.

FIG. 11 shows a process 1100 that will identify a breeding partner that is suited for producing healthy offspring within the breed or sub-breed. That is the method of FIG. 11 will identify within the population of the breed or sub-breed an animal that can provide genetic vigor to the overall breed or sub-breed. This avoids the need to go outside of the breed to get heterogeneity and genetic vigor. The process 1100 begins at 1102 where an animal is selected and for that animal. The process will determine a measure of inbreeding for that animal and this may be measured by the (LOH) loss of heterozygosity. See Jeffrey R. Powell et al.; How Much Does Inbreeding Reduce Heterozygosity? Empirical Results from Aedes aegypti; Am J Trop Med Hyg. 2017 Jan. 11; 96(1):157-158.

The process will optionally generate a B-allele plot for the animal. The process will then analyze the gaps within the sequence indicating heterogeneity and for those gaps, which indicate homogeneity, the process will develop and ideal or a range of ideal genetic sequences with which the animal may be bread. That ideal genetic profile for a mate may be compared to the actual genetic profiles of the existing breeding pool in that breed or sub-breed group and the partner with the best match may be selected for breeding. In some embodiments, the comparison is made to avoid mating one horse with another horse from the same sub-breed where that horse has similar gaps in its B-allele plot. This can avoid propagating a LOH within the sub-breed and provide the heterogeneity that supports continued health and fertility within the sub-breed. In some embodiments, the identification of a breeding partner from the homogeneity plot for a particular animal may be done by veterinarian who is looking at the B plot and comparing it to B plots of candidate mates. It all depends in part on how large the breeding pool is. Some pools are tragically so small that it is difficult to justify mathematical analysis of the homogeneity between different animals. This is particularly true for animals that are rare or moving towards extinction. In any case the method set forth in FIG. 11 will identify a mating pair having an improved chance of producing healthy offspring.

In another aspect it will be understood that disclosed herein are systems for determining a mate for a sub-breed of large animals to provide a breeding process that conserves the sub-breed. To this end, the systems may include a database of genomic information of cohorts of sub-breeds of a large animal population. The system may include processes, including software processes, that can analyze the genomic data to perform principal component analysis as described above. In this case principal component analysis will be used to identify a low dimensional space that measures the difference between the cohorts of known sub-breeds and a reference genome. Processes may also be employed for cluster analysis that will identify with a predetermined level of accuracy the clusters that represent cohorts of large animals that represent the sub-breeds of interest.

The processes may review the genomic information of animals within an identified sub-breed, as these animals which can be found by cluster analysis. Genomic data of respective animals within a particular cluster of sub-breed, now having been quantitatively identified as a member of that sub-breed through use of genomic data, may be analyzed to determine the level of heterogeneity of the particular animal and typically determine an allele plot to understand the particular locations of loss of heterogeneity for a particular animal. A mate may be found within the sub-breed cohort that offers a genetic profile that avoids further loss and propagation of loss of heterogeneity and provides stronger hybrid vigor to provide within the sub-breed an option for a healthy mating to lead to a foal or other juvenile animal that is healthy, fertile, and avoids having been crossbred with a member of a different sub breed. The systems described herein may provide reports that can be provided to breeders to help select mates for conserving the population of a sub breed.

Those skilled in the art will know or be able to ascertain using no more than routine experimentation, many equivalents to the embodiments and practices described herein.

Accordingly, it will be understood that the invention is not to be limited to the embodiments disclosed herein, but is to be understood from the following claims, which are to be interpreted as broadly as allowed under the law.

1. A method for conserving a sub-breed of a large animal, comprising
collecting genetic sequence data from a population of the large animal where the population includes cohorts of the large animal and each large animal in a cohort is known to be in one of the breeds or sub-breeds of the large animal so that the population includes large animals of different sub-breeds,
performing principal component analysis on a measure of genetic variation between a member of the population of large animals and a reference genome,
performing a cluster analysis to determine whether the principal component analysis yields clusters that distinguish between breeds and sub-breeds with a threshold level of accuracy,
selecting a respective one of the large animals in a cohort of a selected sub-breed and
determining a level of heterogeneity for the respective large animal, and
selecting a candidate mate, from the same sub-breed, for the selected large animal based on a heterogeneity profile of the respective large animal to identify a candidate mate having a genomic profile selected to produce offspring with a selected heterogeneity.

2. The method of claim 1, wherein collecting genetic sequence data from a population of the large animal that are known to be in the sub-breed includes identifying a population of the large animal that have been identified with the sub-breed from a comparison of morphological characteristics in a standard set for the sub-breed.

3. The method of claim 1, wherein collecting genetic sequence data from a population of the large animal that are known to be in the sub-breed includes identifying a population of the large animals by comparing parentage breed data wherein the parentage breed data includes information about the morphological characteristics of ancestors of the large animal as compared against morphological standards set for the sub-breed of the large animal.

4. The method of claim 1, wherein collecting genetic sequence data from a population of the large animal that are known to be in the sub-breed includes identifying a population of the large animals by comparing genomic sequence data of a population of the large animal against genomic sequence data associated as a standard for a sub-breed of the large animal.

5. The method of claim 1, wherein performing principal component analysis on a measure of genetic variation between a member large animal of the population of large animals and a reference genome includes comparing gene sequence data for the member large animal against gene sequence data of the reference animal and identifying differences between the two sequences.

6. The method of claim 1, wherein performing a cluster analysis includes generating a dendrogram to determine a relationship between members of sub-breeds for allocating animals to clusters.

7. The method of claim 1, wherein performing a cluster analysis includes a nearest neighbor analysis.

8. The method of claim 1, wherein selecting a respective one of the large animals in a cohort determining the level of heterogeneity includes analyzing the genome of the respective large animal considering the paternal and maternal genetic data and making a fractional analysis of identity of paternal and maternal genetic data to measure a level of inbreeding.

9. The method of claim 1, wherein selecting a mate having a genomic profile selected to produce healthy offspring with a selected heterogeneity includes selecting a mate having greater heterogeneity at a select sequence location than the selected large animal.

10. The method of claim 1, wherein selecting a mate, from the same sub-breed, for the selected large animal based on a heterogeneity profile of the selected large animal includes selecting a large animal having a genomic profile selected to produce healthy offspring capable of breeding.

11. The method of claim 1, further comprising generating a B-allele plot for the selected large animal to provide a heterogeneity profile for the selected large animal and generating a B-allele plot for the candidate mate to provide a heterogeneity profile for the candidate mate and determining the quality of the candidate mate as a function of the similarity of the heterogeneity profiles of the candidate mate and the selected large animal.

12. The method of claim 11, wherein determining the quality of the candidate mate as a function of the similarity of the heterogeneity profiles of the candidate mate includes selecting a candidate mate to achieve an acceptable level of heterozygosity of a foal.

13. The method of claim 1, further including analyzing members of a sub-breed cluster to determine a genetic inbreeding coefficient for the sub-breed.

14. The method of claim 13, wherein analyzing members of a sub-breed cluster to determine a genetic inbreeding coefficient includes generating violin graphs indicating a genetic inbreeding coefficient.

15. The method of claim 1, wherein the large animal is any of a horse, a camel and cattle.
